(19) United States
(12) Patent Application Publication
(10) Pub. No.: US 2018/0207023 A1
(43) Pub. Date: Jul. 26, 2018

(54) APPARATUS FOR LOADING, INSERTION AND PLACEMENT OF AN INTRAUTERINE CONTRACEPTIVE DEVCE AND PROCESS THEREOF

(71) Applicant: PREGNA INTERNATIONAL LIMITED, MUMBAI, Maharashtra (IN)

(72) Inventors: Deepak Mehra, Mumbai (IN); Mukul Taparia, Mumbai (IN)

(73) Assignee: PREGNA INTERNATIONAL LIMITED, Mumbai (IN)

(21) Appl. No.: 15/745,579

(22) PCT Filed: Sep. 8, 2015
(86) PCT No.: PCT/IN2015/000351

§ 371 Date: Jan. 17, 2018

(30) Foreign Application Priority Data
Jul. 21, 2015 (IN) 2750/MUM/2015

(51) Int. Cl. A61F 6/18

(52) U.S. Cl. CPC A61F 6/18; A61F 6/144

(57) ABSTRACT

A process and an apparatus for loading, insertion and placement of an intrauterine contraceptive device (IUD) for its proper and hygienic placement in the uterus, the apparatus in a sterile pouch having a transparent cover and a base; the apparatus having a loader with a passage having a first opening and a second opening running along its elongated body, a graduated tube mounted onto loader through its second opening, an IUD and a push rod miming inside the graduated tube. The IUD is parked in the loader in an expanded condition, required to maintain inside the uterus. The loader has a guide means forming the front portion of the loader and pusher means forming rear portion of the loader. A platform connects the guide means and the pusher means of the loader. The passage runs along an elongated body of the loader.

### FIELD OF THE INVENTION

The present invention relates to an apparatus for intrauterine contraceptive device, commonly known as IUD or IUS. Particularly, the present invention relates to an apparatus for preparation of the IUD/IUS for a proper and hygienic placement in uterus, and process of placement of IUD/IUS in uterus.

### BACKGROUND OF THE INVENTION

An intrauterine contraceptive device, commonly abbreviated as an IUD; also known as an intrauterine system and abbreviated as IUS, is a birth control device, which is placed in uterus of a woman. IUDs have been known since several decades, and are also popularly known as “Copper-T”, since originally, the device only had copper wire or sleeves or both on a thermoplastic or non-metallic frame. The intrauterine devices or intrauterine systems (hereinafter referred to as IUDs) currently available have copper, copper-silver combination, hormonal drugs, supplementary drugs et cetera and are available in various shapes, most common being T-shaped; and sized to fit in uterus of different size of different women.

The IUD is required to be in some commensurate shape with respect to the uterine cavity, like the shape of English letter “T”, so as to stay in the uterus appropriately. However, the IUD cannot be inserted in “T” form, because the opening of the cervix is narrow. The arms of the IUD, forming “T” shape are folded either upwards or downwards so that IUD can be contained in a narrow tube which then can be inserted in the uterus.

The process of folding the arms of the IUD and containing the IUD in an apparatus used for insertion, commonly a narrow tube, is defined as loading of IUD. The process of inserting the IUD with the aid of the narrow tube up to the fundus is defined as insertion of IUD.

The process of releasing the folded arms of the IUD so as to allow the IUD to regain its “T” shape, and freeing the IUD from the narrow tube at an appropriate place in the uterus is defined as placement of IUD.

EP 1263361 B1 discloses a device for inserting a T-shaped intra-uterine device where the folding of the arms of the IUD is done in the upward direction, that is, away from the stem of the IUD. Inserter tube is provided with the slots to retain the aims of IUD. A tip of a part of the insertion tube which is to enter the cervix is frustoconical in shape, formed by two petals, created by incising the insertion tube, and the petals are expected to be flexible to open and close.

Folding of the arms of the IUD downwards, that is, towards the stem of the IUD, is very different. Patent No: U.S. Pat. No. 4,249,525 discloses an instrument for loading an IUD into the tube. The instrument has opposite side walls having channels that exert force to fold the arms as the IUD is pushed into the apparatus. The end wall stops further movement and the side walls flex as the tube is forced in. It is apparent that significant manual maneuvering is called for in order to achieve desired folding of the arms of IUD.

Patent no U.S. Pat. No. 5,842,474 A discloses another method of loading the IUD by use of movable folding device that acts on the IUD which is releaseably positioned in a component termed stage. The manual action here appears to be simpler.

Patent No U.S. Pat. No. 4,549,652A discloses an IUD package wherein arms of an IUD can be manipulated inside the package. The package is provided with the cavity wherein IUD is pushed by the insertion tube and this result in folding of arms of the IUD. The arms of IUD are then placed within the insertion tube. The loading process is thus managed with the fingers of the person from outside the package; the challenge being to orchestrate proper folding of the arms of the IUD, which are delicate.

U.S. Pat. No. 4,143,656A discloses an instrument where an insertion tube has housing for IUD, which is essentially another tube of larger diameter, and wherein the arms of the IUD fold, in between the housing and the insertion tube, while the stem of the IUD remains in the insertion tube. The instrument is provided with a handle which is a part of an insertion tube; the locking arrangement is provided at the handle for irreversibly releasing arms of the IUD from the housing.

Patent application no: US20130068234A1 discloses a tool for loading, inserting and releasing of an IUD in uterus. The tool comprises of shaft having a loader, an inserter tube, an IUD and a plunger. A slot running all along the shaft with an insertion tube and push rod running inside said slot. A knob is provided on insertion tube to push inserter tube and push rod in backward or forward direction. The tool apparently is an integrated apparatus focusing on aesthetical appeal and automated “feel” through knob operation, which are back and forth, for steps of loading, inserting and release.

The instruments or apparatus known, thus, either have complex loading components and manufacturing process, or there are number of skillful steps involved in loading which may confuse the surgeon. Therefore there is a need to invent an instrument which can simplify the loading, insertion and placement of IUD in uterus.

The present invention overcomes these difficulties.

### OBJECTIVE OF THE INVENTION

The objective of the invention is to simplify loading of an IUD safely into an insertion tube.

Another objective is to invent an apparatus for loading, insertion and placement of IUD, which is simple and economical.

Yet another objective of the invention is to provide an apparatus which comprises of minimum of moving and separate parts.

Yet another objective of the invention is to provide an apparatus which is friendly to use, i.e. without experienced surgeons having to be specifically trained for.

Yet another objective is to invent an apparatus which does not cause additional discomfort to woman while placing the IUD in uterus.

### STATEMENT OF INVENTION

The present invention is an apparatus for loading, insertion and placement of an intrauterine contraceptive device (IUD) and process thereof. ‘T’ shaped IUD is considered for disclosure of our invention.

The apparatus comprises of a loader slidably mounted on a graduated tube, an IUD and a push rod running inside the graduated tube. The IUD is parked in the loader in an expanded condition, that is, in a “T” shape. The apparatus is packaged in a sterile pouch with a transparent cover and a base.

The loader comprises of an elongated body. A guide means forms a front portion of the loader and a pusher means forms a rear portion of the loader. A platform connects the guide means of the loader to the pusher means of the loader. A passage runs along the elongated body of the loader.

The guide means has a stopping face at its front end which forms a part of the loader, acts as a measurement reference for sounding and also acts as a stopper of the loader at cervical os. The guide means has an IUD retainer in a vicinity of its rear end.

The IUD retainer has a cantilever; the cantilever is formed by providing slits on an upper surface of the loader, located in the vicinity of the rear end of the guide means towards the pusher means. The cantilever has teeth at its free end.

The teeth of the IUD retainer perform any or all of the following three functions:
1. hold the arms of an IUD while in expanded condition, in the pair of C-shaped exits,
2. act as an initial stopper for the graduated tube.
3. prevent the stem of the IUD from bending during the loading process.

The passage has a first opening on the stopping face, a second opening at the rear end of the pusher means, and a pair of C-shaped exits on either sides at the rear end of the guide means of the loader. The passage within the guide means that is, between the first opening of the guide means and the pair of C shape exit at rear end of the guide means is called as a negotiation passage. The passage within the pusher means i.e. between front opening of the pusher means and second opening of the pusher means is called as a tubular passage. The passage runs along the elongated body of the loader from the first opening of the guide means to the second opening of the pusher means.

The negotiation passage is a converging curved shaped, from C-shaped exit till the first opening on the stopping face. The negotiation passage begins from the pair of C-shaped exits on either side of the loader and converge into the first opening whose area of cross-section is substantially same as that of an area of cross section of the second opening of the pusher means.

The first opening has the non-circular cross-section with the top flattened and the bottom slightly projected enables a loading, that is, folding of arms of IUD such that the arms are always towards the upper surface of the loader and the stem of the IUD is always towards the lower surface of the loader.

The pusher means has an opening at its rear end, forming the second opening of the passage. The graduated tube is slidably mounted onto the loader through the second opening of the pusher means. The guide means and the pusher means are provided with a holding face on either side of the loader. The holding faces allow gripping of the loader, during the loading and insertion of the IUD. The IUD is parked on the platform, the arms of the IUD getting accommodated in the pair of C-shaped exits.

The graduated tube is dimensioned to accommodate the arms and the stem of the IUD such that the top of the orifice is flattened and the bottom becomes slightly projected, which is nearly a triangular formation. The graduated tube is provided with measurement markings towards its proximal end.

A push rod has a handle. The push rod freely moves in the graduated tube. The push rod optionally has a localized projection which rubs inside the graduated tube & thereby facilitates retaining the push rod at any position within and with respect to the graduated tube.

When the apparatus is in a sterilized transparent pouch as received, the graduated tube is mounted in the loader up to the platform, such that it partly remains in the tubular passage of the passage while remaining portion of the graduated tube extends outside from the second opening on the rear end of the pusher means. The graduated tube is provided with a measurement markings on its outer surface, to facilitate the positioning of stopping face. The IUD is parked at the platform. The stem partially and the strings fully are accommodated inside the graduated tube. The push rod that runs inside the graduated tube, is kept such that a tip of the push rod stays slightly away from, or barely touches, the bulging end of the stem of the IUD.

The process of loading of the IUD is carried out as follows:
1 Partially separating a transparent cover from the base of the pouch, to be able to access and hold the handle of the push rod and the graduated tube.
2 Holding both the holding faces of the loader with one hand from outside of the transparent cover.
3 Pushing in the graduated tube till its proximal end touches the edge of the teeth of the IUD retainer.
4 Pushing in the push rod in the graduated tube. The tip of the push rod in turn pushes the bulging end of the IUD that rests on the platform of the loader. The IUD starts entering the negotiation passage. As a result of the pushing by the push rod, the arms of the IUD negotiate their shape and consequently, the arms of the IUD now starts folding. At this stage the position ‘P’ of the handle of the push rod changes to a position ‘P1’. The push rod is pushed inside till the smooth curve of the IUD just starts projecting out from the first opening from the stopping face of the loader. At this instance, a root of the handle of the solid rod touches a distal end of the graduated tube and at this position the handle of the solid rod attains a position P2. Alternatively, either of the conditions, that is, either the projecting IUD or the P2 position are achieved to signify and ensure that the arms of the IUD are in folded condition and ready to be held in the graduated tube.
5 Advancing the graduated tube towards the first opening with a force ‘F’. Due to the applied force F acting on the edge of the teeth of the cantilever, the IUD retainer is lifted upwards with raised cantilever providing a way for the graduated tube to move into the negotiation passage. The arms of the IUD enter the graduated tube, since the arms of the IUD are now substantially in the same orientation as the stem of the IUD, which is partially already in the graduated tube.
6 Pushing the graduated tube along with the IUD with folded arms further towards the first opening provided at the front end of the guide means such that an orifice (8) at a proximal end of the graduated tube comes out of the first opening along with a captured IUD out of the loader. This completes the process of process of loading.

The stopping face is then adjusted on the measurement markings of the graduated tube to a predetermined position for the insertion according to the depth of the uterus in accordance with the sounding measurement carried out independently as is known.

The process is carried out while the apparatus is inside the partially open pouch. The graduated tube, along with the IUD loaded, is inserted inside the uterus till stopping face touches the external cervical os (53) of the woman.

The inserted graduated tube is then gradually withdrawn; keeping the push rod steady, to first free the arms of the IUD and then release the IUD from the graduated tube, while the IUD stays in the position due to the tip of the push rod firmly butting on the bulging end of the stem of the IUD.

The two-stage withdrawal—first only the graduated tube keeping the push rod steady, and then the apparatus, ensures that the IUD is not disturbed by the withdrawal of the graduated tube.

### BRIEF DESCRIPTION OF DRAWINGS

FIG. 1 shows an apparatus in a sterilized transparent pouch.

FIG. 2A shows an IUD in an expanded condition i.e. “T” shape.

FIG. 2B shows the IUD with arms folded above the stem.

FIG. 3 is a perspective view of the apparatus having a loader, a graduated tube with a push rod therein and the IUD.

FIG. 4 gives two perspective views of the loader.

FIG. 5 provides a detailed construction of an IUD retainer with IUD parked on a platform of the loader, while FIG. 5A is a side view of the loader, showing an angle between an edge of the teeth of the IUD retainer, and the platform, of the loader.

FIG. 6 is a top sectional view of the loader showing a construction of a passage inside the loader, while FIG. 6A shows several sections of a negotiation passage.

FIG. 7 is a front sectional view of the loader showing shape of the front opening of the loader.

FIG. 7A is a sectional view of the loader with the folded IUD and the graduated tube in the vicinity of the first opening, while FIG. 7B shows a perspective view of folded IUD with the arms above the stem.

FIG. 8 is perspective view of a graduated tube

FIG. 9 is a perspective view of a push rod.

FIG. 10 is a top view of the apparatus with the IUD parked in the loader, ready for loading, along with the graduated tube and a handle of the push rod at a position P.

FIG. 11 is a top view of the apparatus, shows the IUD pushed into a negotiation passage with the handle of the push rod moved from the position P to a position P1.

FIG. 12 is a top view of the apparatus, showing arms of the IUD folded inside the negotiation passage and handle of the push rod attain a position P2.

FIG. 13 shows IUD with folded arms inside the graduation tube when a force F is applied.

FIG. 13A shows the graduation tube advanced towards the first opening leading to an uplifting of the cantilever and teeth when the force F is applied.

FIG. 14 shows the graduated tube along with the IUD loaded, pushed outside the front opening of the loader.

FIG. 14A shows the stopping means adjusted with respect to the measurement markings on the graduated tube.

FIG. 15 shows the graduated tube with the IUD, inserted in a uterus.

FIG. 16 shows the IUD with the arms in expanded condition, placed in the uterus.

FIG. 17 shows another embodiment of the apparatus with stopping means separate from the loader.

FIG. 18 shows a perspective view of another embodiment of the loader having first opening of the loader with a circular cross section; and a sectional view of the apparatus showing a proximal end of the graduated tube with a corresponding circular cross section.

FIG. 19 shows a perspective view of another embodiment of the loader without an IUD retainer.

FIG. 20 shows perspective view of yet another embodiment of the loader with the guide means and the pusher means of the loader as separate components.

### DETAILED DESCRIPTION OF INVENTION

The preferred embodiment of an apparatus and a loader as per our invention shall now be described in detail, with reference to the accompanying drawings. The terms and expressions which have been used here are merely for description but the invention can be worked with several variations and the terms and expressions should not be construed to be limiting the invention in any way.

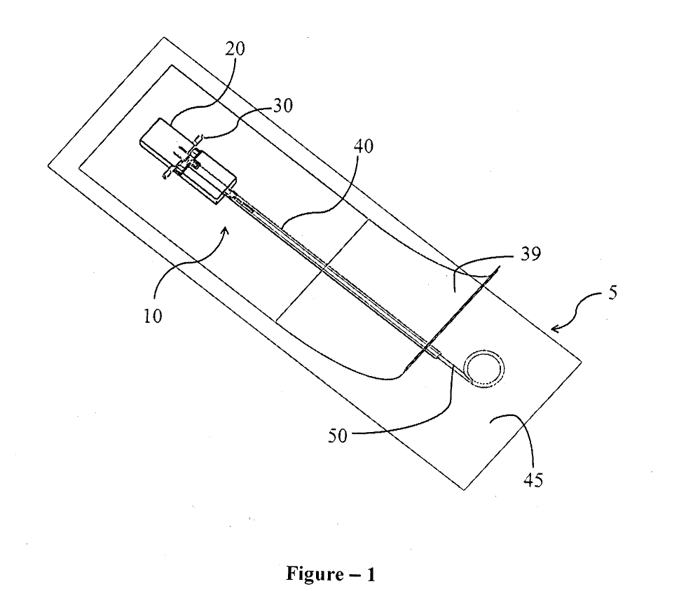

According to a preferred embodiment of present invention, FIG. 1 shows an apparatus (10) having a loader (20) slidably mounted on a graduated tube (40), an IUD (30) and a push rod (50) running inside the graduated tube (40). The IUD (30) is parked in the loader (20) in an expanded condition, that is, in a “T” shape which it is required to maintain inside a uterus. The apparatus (10) is packaged in a sterile pouch (5) with a transparent cover (39) and a base (45).

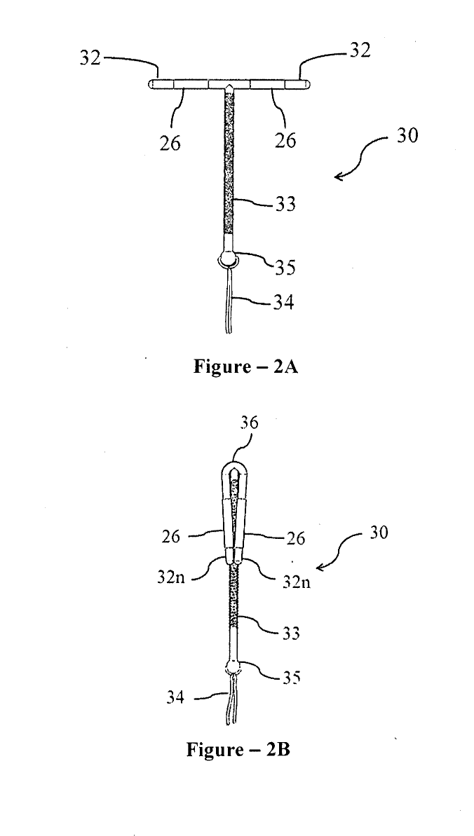

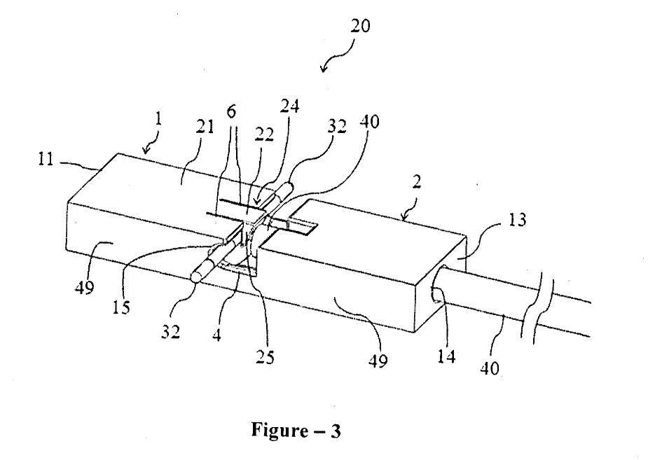

As shown in FIG. 2A, the IUD (30) comprises of a frame which is made of a thermoplastic material. The frame has a vertical stern (33) and an arms (32) forming the “T” shape, which is the expanded condition of the IUD (30). A copper wire (not numbered) is wrapped around the stern (33). The wire may be of combination metals as well. The arms (32) have copper sleeves (26) thereon; however not all IUDs may have such sleeves (26). Prior to inserting the IUD (30) in the uterus (60), the arms (32) of the IUD (30) should be folded so that the folded arms (32) and the stem (33) of the IUD (30) can be contained in a graduated tube (40) used for insertion. The folding of arms (32) to folded arms (32n) result into formation of a smooth curve (36), as shown in FIG. 2B. The smooth curve (36) facilitates entry of the IUD (30) in the external cervical os. Referring to FIG. 2A and 2B, a bulging end (35) of the stem (33) has a string or strings (34) attached to it. The string or strings (34) assist in easy removal of the IUD (30) from the uterus (60) after use or whenever needed, as also facilitate self-ensuring a woman of the presence of IUD by “feeling” the strings with her finger(s).

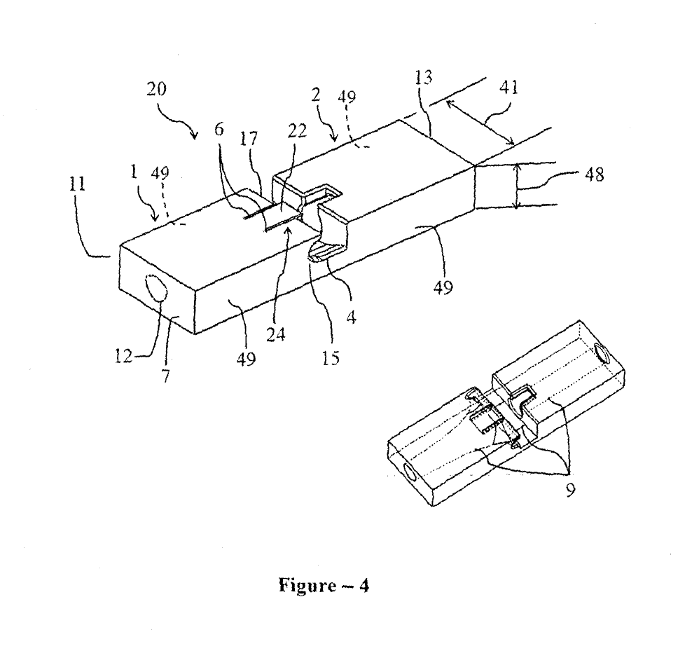

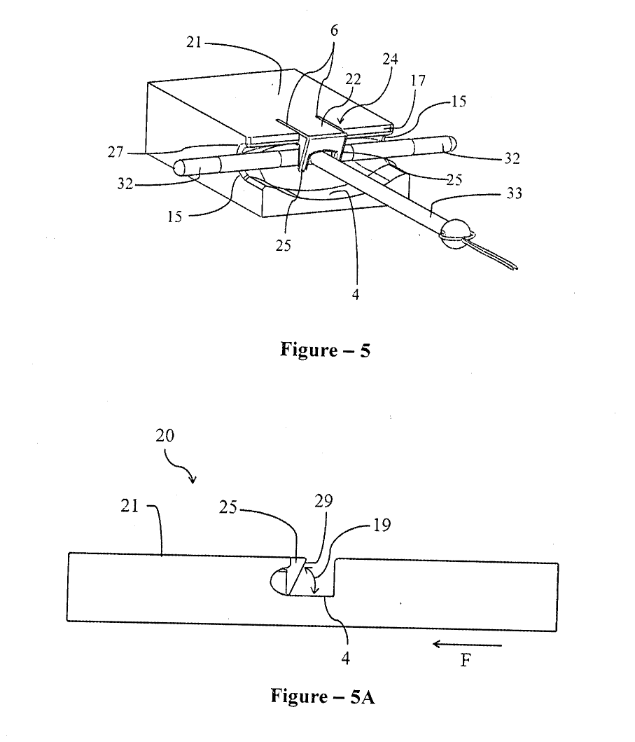

FIG. 3 and FIG. 4 show the loader (20) having an elongated body. A guide means (1) forms a front portion of the loader (20) and a pusher means (2) forms a rear portion of the loader (20). A platform (4) connects the guide means (1) of the loader (20) to the pusher means (2) of the loader (20). A passage (9) runs along the elongated body of the loader (20).

The guide means (1) has a stopping face (7) at its front end (11) which forms a part of the loader (20), acts as a measurement reference for sounding and also acts as a stopper of the loader (20) at cervical os. The guide means (1) has an IUD retainer (24) in a vicinity of its rear end (17).

As shown in FIG. 5 and FIG. 5A, the IUD retainer (24) has a cantilever (22). The cantilever (22) is formed by providing slits on an upper surface (21) of the loader (20), located in the vicinity of the rear end (17) of the guide means (1) towards the pusher means (2). The cantilever (22) having teeth (25) at its free end. The cantilever (22) normally remains in the same plane as the upper surface (21) of the loader (20). An edge (29) of the teeth (25) is at an angle (19) of 20 to 70 degrees with respect to the platform (4). The cantilever (22) can be pushed up when a force F, parallel to the platform (4) or the upper surface (21) acts on an edge (29) of the teeth (25).

The teeth (25) of the IUD retainer (24) perform any or all of the following three functions:
1. hold the arms (32) of an IUD (30) while in expanded condition, in the pair of C-shaped exits (15),
2. act as an initial stopper for the graduated tube (40).
3. prevent the stern (33) of the IUD (30) from bending during the loading process.

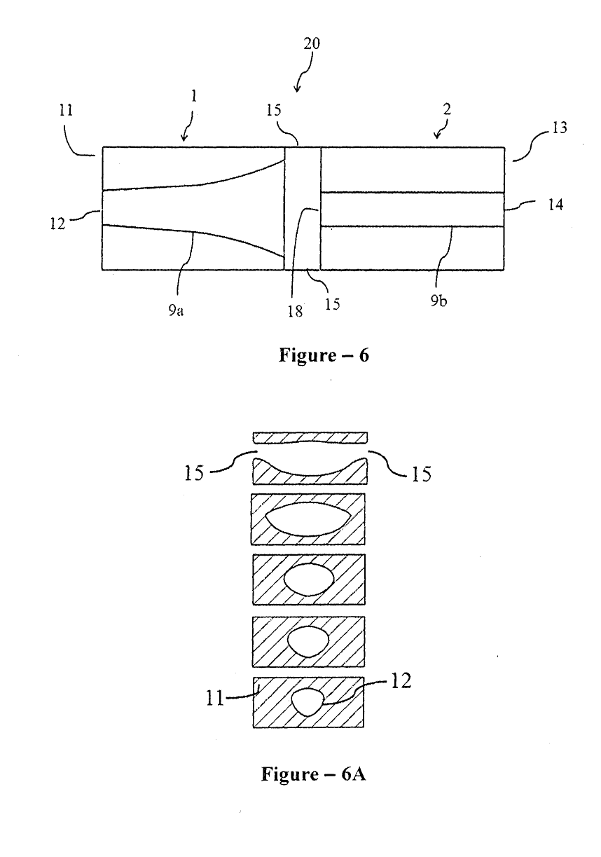

The passage (9) has a first opening (12) on the stopping face (7), a second opening (14) at the rear end of the pusher means (13), and a pair of C-shaped exits (15) on either sides at the rear end (17) of the guide means (1) of the loader (20), as seen in FIG. 6.

The passage (9) within the guide means (1) that is, between the first opening (12) of the guide means (1) and the pair of C shape exit (15) at rear end (17) of the guide means (1) is called as a negotiation passage (9a), whereby the folding of the arms (32) of the IUD (30) takes place by way of the arms (32) of the IUD (30) negotiating its way forward and get bent as a force pushes the IUD (30) into the negotiation passage (9a). The passage (9) within the pusher means (2) i.e. between front opening (18) of the pusher means (2) and second opening (14) of the pusher means (2) is called as a tubular passage (9b). The passage (9) runs along the elongated body of the loader (20) from the first opening (12) of the guide means (1) to the second opening (14) of the pusher means (2) as shown in FIG. 6.

As seen in FIG. 6, the negotiation passage (9a) is a converging curved shaped, from C-shaped exit (15) till the first opening (12) on the stopping face (7). The negotiation passage (9a) begins from the pair of C-shaped exits (15) on either side of the loader (20) and converge into the first opening (12) whose area of cross-section is substantially same as that of an area of cross section of the second opening (14) of the pusher means (2), however, is a non-circular, a top is flattened (46) and a bottom is slightly projected (47) as shown in FIG. 6A, the FIG. 6A also showing intermediate shape of the negotiation passage (9A).

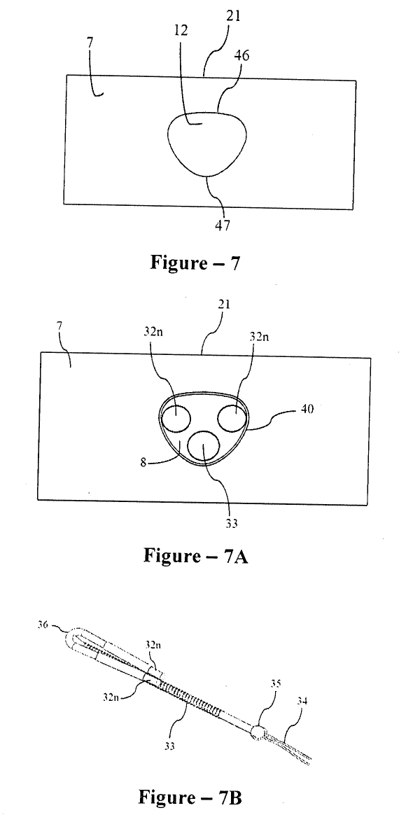

The first opening (12) which thus has the non-circular cross-section with the top flattened (46) and the bottom slightly projected (47) enables a loading, that is, folding of arms (32) of IUD (30) such that the arms (32n) are always towards the upper surface (21) of the loader (20) and the stem (33) of the IUD (30) is always towards the lower surface (not numbered) of the loader (20) as shown in FIG. 7 and FIG. 7A. Such consistent folding of the IUD (30), that is, the arms (32) of the IUD (30) always towards the upper surface (21) of the loader (20) and the stem (33) of the IUD (30) always towards the lower surface of the loader (20) gives additional confidence to the surgeon while loading the IUD (30) that the IUD (30) is indeed loaded properly as shown in FIG. 7B.

As FIG. 3 shows, the pusher means (2) has an opening at its rear end (13), forming the second opening (14) of the passage (9). The graduated tube (40) is slidably mounted onto the loader (20) through the second opening (14) of the pusher means (2). The guide means (1) and the pusher means (2) are provided with a holding face (49) on either side of the loader (20). The holding faces (49) allow gripping of the loader (20), during the loading and insertion of the IUD (30).

The IUD (30) is thus parked on the platform (4), the arms (32) of the IUD (30) getting accommodated in the pair of C-shaped exits (15).

As shown in FIG. 8, seen with FIG. 7, the graduated tube (40) is a thin walled, flexible, circular, hollow open cylinder, having an inner cross section dimensioned to accommodate the arms (32) and the stem (33) of the IUD (30) such that the top of the orifice (8) is flattened (46) and the bottom becomes slightly projected (47), which is nearly a triangular formation. The graduated tube (40) is provided with measurement markings (43) towards its proximal end (23). The cross section of the first opening (12) of the guide means (1) is dimensioned such that the orifice (8) of the graduated tube (40) takes a shape complementary to the shape of the first opening (12). Thus, the orifice (8) of the graduated tube (40) consistently accommodates the arms (32) and the stem (33) of the IUD (30) as shown in FIG. 7A.

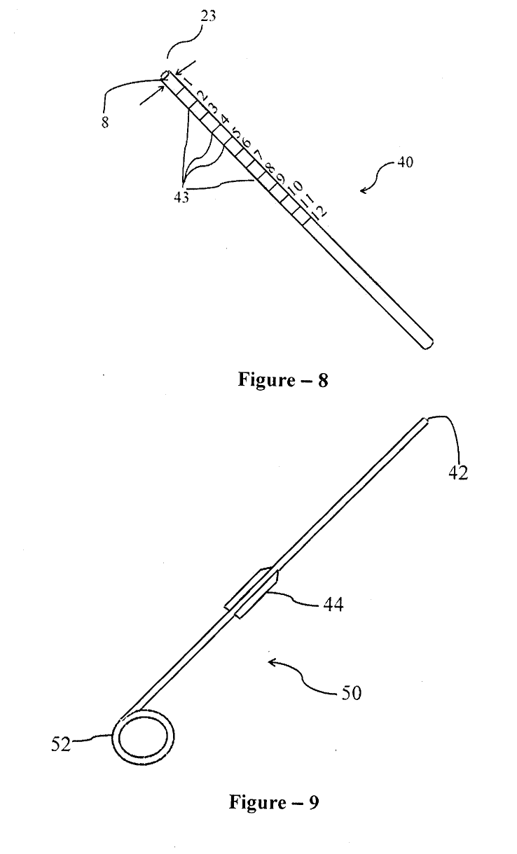

As shown in FIG. 9, a push rod (50) has a handle (52). The push rod (50) freely moves in the graduated tube (40). The push rod (50) optionally has a localized projection (44) which rubs inside the graduated tube (40) & thereby facilitates retaining the push rod (50) at any position within and with respect to the graduated tube (40).

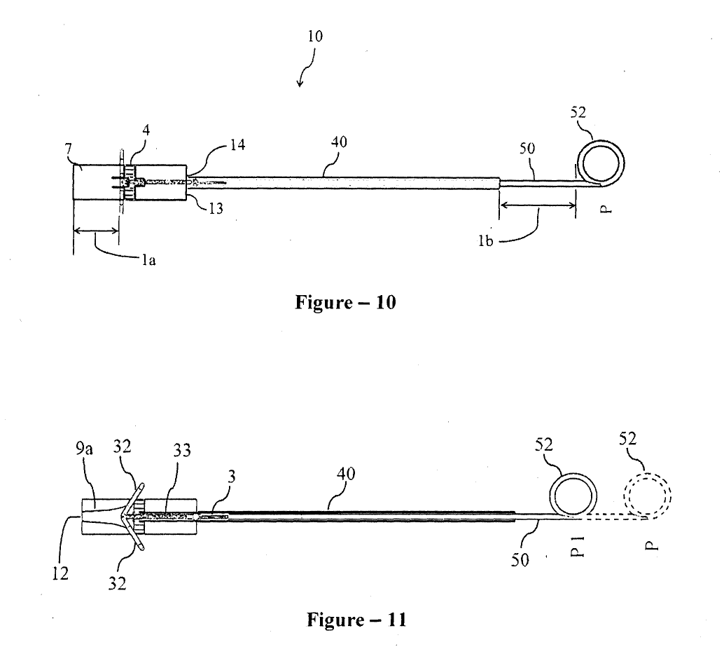

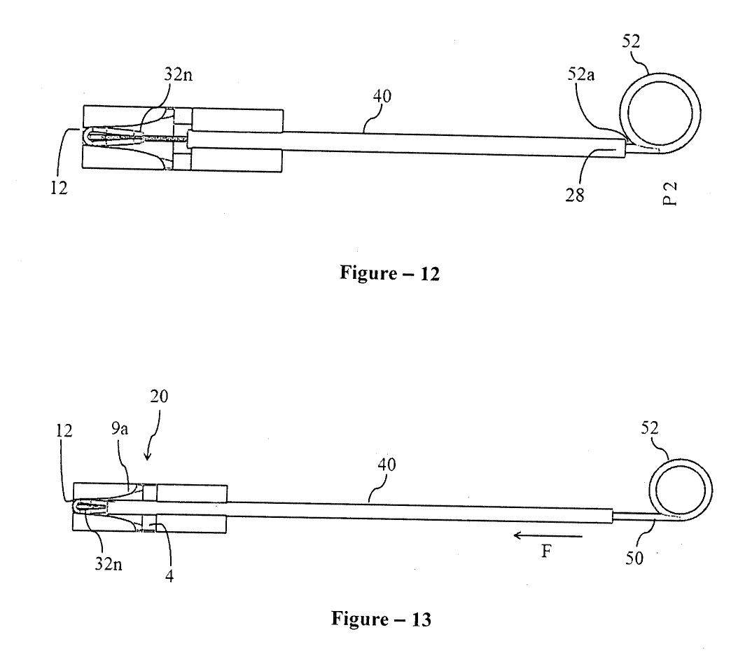

When the apparatus (10) is in a sterilized transparent pouch (5) as received, the graduated tube (40) is mounted in the loader (20) up to the platform (4), such that it partly remains in the tubular passage (9b) of the passage (9) while remaining portion of the graduated tube (40) extends outside from the second opening (14) on the rear end (13) of the pusher means (2). The graduated tube (40) is provided with a measurement markings (43) on its outer surface, to facilitate the positioning of the stopping face (7). The IUD (30) is parked at the platform (4). The stem (33) partially and the strings (34) fully are accommodated inside the graduated tube (40). The push rod (50) that runs inside the graduated tube (40), is kept such that a tip (42) of the push rod (50) stays slightly away from, or barely touches, the bulging end (35) of the stem (33) of the IUD (30) as shown in FIG. 1, FIG. 10 and FIG. 11.

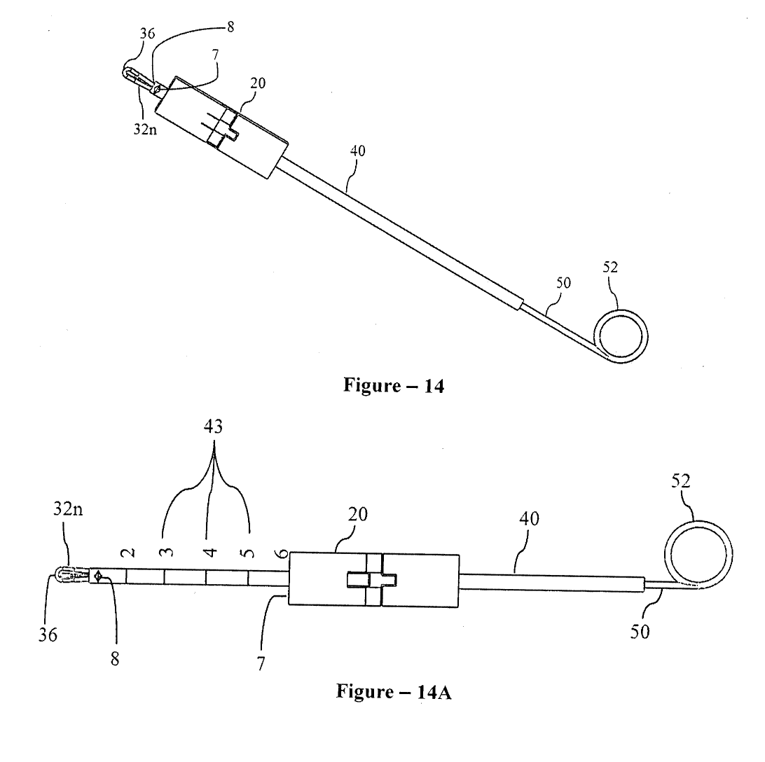

FIG. 1 and FIG. 10 to FIG. 16 show a process of loading of an IUD (30) in the graduated tube (40) and a process of insertion and placement of the IUD (30) in uterus (60). Loading of the IUD (30) is carried out as follows:
Partially separating a transparent cover (39) from the base (45) of the pouch (5), to be able to access and hold the handle (52) of the push rod (50) and the graduated tube (40).
Holding both the holding faces (49) of the loader (20) with one hand from outside of the transparent cover (39).

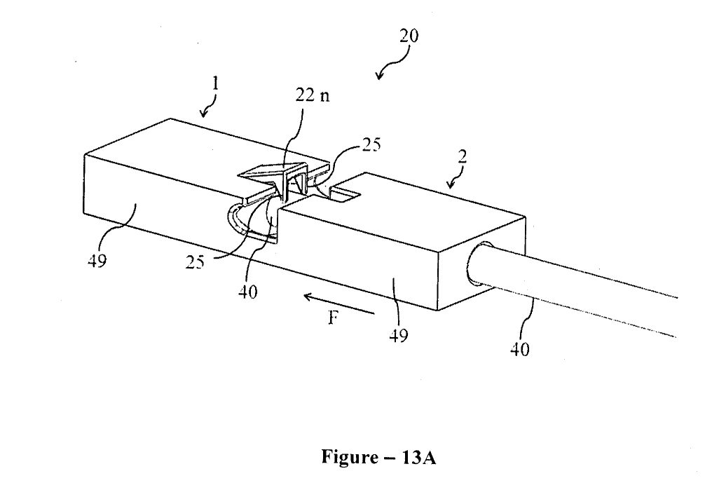

Pushing in the graduated tube (40) till its proximal end (23) touches the edge (29) of the teeth (25) of the IUD retainer (24).
Pushing in the push rod (50) in the graduated tube (40). The tip (42) of the push rod (50) in turn pushes the bulging end (35) of the IUD (30) that rests on the platform (4) of the loader (20). The IUD (30) starts entering the negotiation passage (9a) but the arms (32) of the IUD (30) face minimal resistance due to the converging curved shape of the negotiation passage (9a). As a result of the pushing by the push rod (50), the arms (32) of the IUD (30) negotiate their shape and consequently, the arms (32) of the IUD (30) now starts folding. At this stage the position ‘P’ of the handle (52) of the push rod (50) changes to a position ‘P1’, as shown in FIG. 11. During the folding of arms (32) of the IUD (30), the IUD retainer (24), due to the teeth (27) of the cantilever (22) does not allow IUD (30) to bend sideways. The push rod (50) is pushed inside till the smooth curve (36) of the IUD (30) just starts projecting out from the first opening (12) from the stopping face (7) of the loader (20).
At this instance, a root (52a) of the handle (52) of the solid rod (50) touches a distal end (28) of the graduated tube (40) and at this position the handle (52) of the solid rod (50) attains a position P2, as shown in FIG. 12. This position is achievable by coordinating the length dimension (1a) of the guide means (1) with the differential distance (1b) between the distal end (28) of the graduated tube (40) and the root (52a) of the handle (52) of the push rod (50), when the tip (42) of the push rod (50) touches the bulging end (35) of the IUD (30) as shown in FIG. 10.
Alternatively, either of the conditions, that is, either the projecting IUD (30) or the P2 position are achieved to signify and ensure that the arms (32n) of the IUD (30) are in folded condition and ready to be held in the graduated tube (40).
Advancing the graduated tube (40) towards the first opening (12) with a force ‘F’. Due to the applied force F acting on the edge (29) of the teeth (25) of the cantilever (22), the IUD retainer (24) is lifted upwards with raised cantilever (22n) providing a way for the graduated tube (40) to move into the negotiation passage (9a). The arms (32n) of the IUD (30) enter the graduated tube (40), since the arms (32n) of the IUD (30) are now substantially in the same orientation as the stem (33) of the IUD (30), which is partially already in the graduated tube (40).
Pushing the graduated tube (40) along with the IUD (30) with folded arms (32n) further towards the first opening (12) provided at the front end (11) of the guide means (1) such that an orifice (8) at a proximal end (23) of the graduated tube (40) comes out of the first opening (12) along with a captured IUD (30) out of the loader (20).
   This completes the process of process of loading.

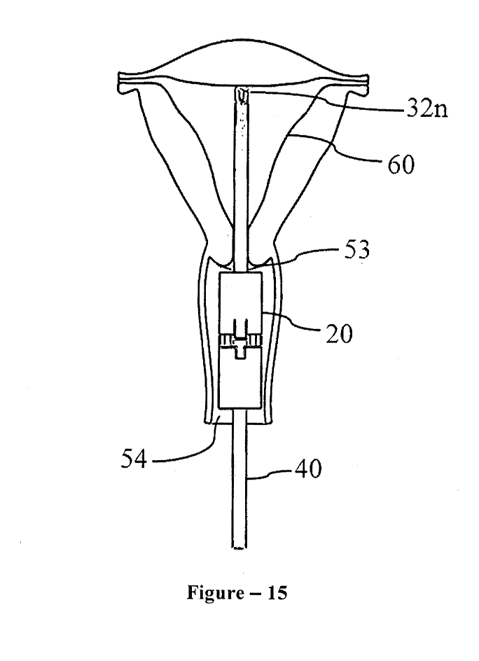

The stopping face (7) is then adjusted on the measurement markings (43) of the graduated tube (40) to a predetermined position for the insertion according to the depth of the uterus (60) in accordance with the sounding measurement carried out independently as is known as shown in FIG. 14A.

The process thus far is carried out while the apparatus (10) is inside the partially open pouch (39, 45). Consequently, the IUD (30) is loaded without human touch to avoid possible infection during insertion.

The graduated tube (40), along with the IUD (30) loaded, is inserted inside the uterus (60) till stopping face (7) touches the external cervical os (53) of the woman.

The inserted graduated tube (40) is then gradually withdrawn; keeping the push rod (50) steady, to first free the arms (32n) of the IUD (30) and then release the IUD (30) from the graduated tube (40), while the IUD (30) stays in the position due to the tip (42) of the push rod (50) firmly butting on the bulging end (35) of the stem (33) of the IUD (30).

Consequent to a well-known “memory” feature of the thermoplastic materials, the arms (32) of the IUD (30) return back to the expanded condition, that is, ‘T’ shape, leading to placement of IUD (30) in the uterus (60). The push rod (50) along with the graduated tube (40) is then withdrawn out of the uterus (60).

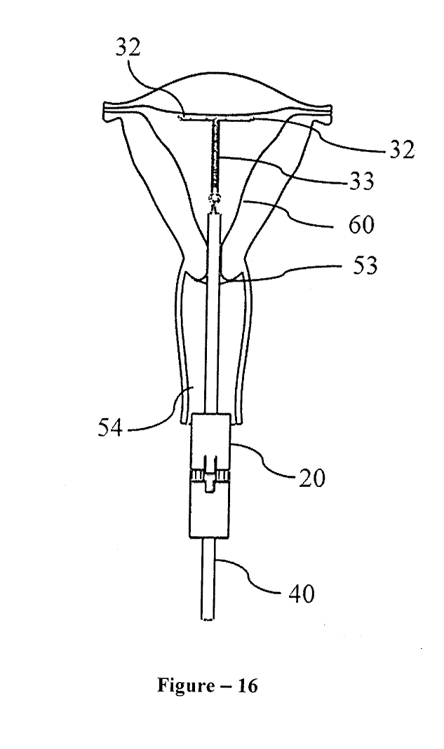

This two-stage withdrawal—first only the graduated tube (40) keeping the push rod (50) steady, and then the apparatus (10), ensures that the IUD (30) is not disturbed by the withdrawal of the graduated tube (40), as shown in FIGS. 15 and 16.

Since the loader (20) is required to enter the vaginal cavity (54) in order to ensure that the stopping face (7) touches the cervical os, it is important to ensure that an overall width (410) and an overall height (480) of the loader are constrained to a minimum size, which can easily enter the vaginal cavity (54), after the vaginal cavity (54) is stretched open for an IUD (30) implantation, as is a known practice. The overall width (41) of the loader (20) is preferably less than 18 mm and the overall height (48) of the loader (20) is less than 12 mm.

Construction of the different parts of the loader (20) is possible in different ways, for example, the passage (9) could be or by providing a straight but angular slant instead of converging curve as negotiation passage (9a), conceptually and eventually leading to folding of the arms (32) of IUD at it is pushed inside the loader (20).

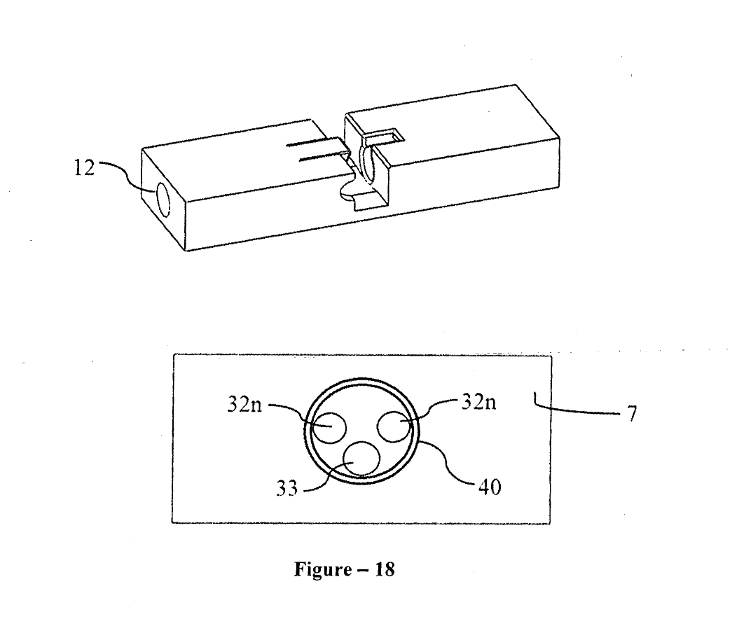

The negotiation passage (9a) which begins as two C-shaped exits (15) on either side of the loader (20) and converges into a first opening (12) whose area of cross-section is substantially same as that of the second opening (14) of the pusher means can be circular, as another embodiment and as shown in FIG. 18, whereby the proximal end of the graduated tube (40) thus remains circular is it projects out of the stopping face (49) with loaded IUD (30).

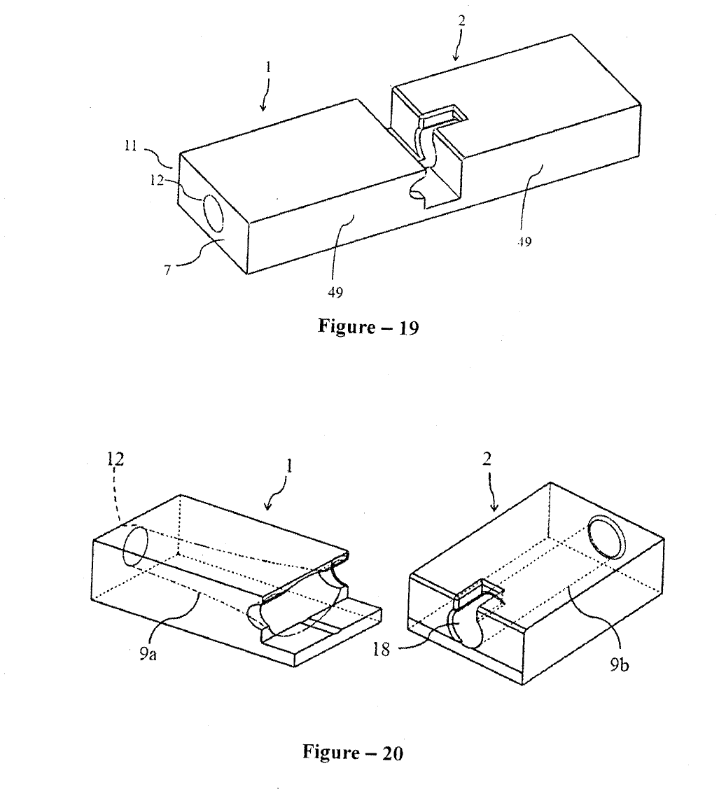

The loader (20) as another embodiment can be without an IUD retainer (24) as shown in FIG. 19, or the IUD retainer (24) can be a separate component. In case of the loader (20) without the IUD retainer (24), loading of the IUD (30) is carried out as follows:
1 Partially separating a transparent cover (39) from the base (45) of the pouch (5), to be able to access and hold the handle (52) of the push rod (50) and the graduated tube (40).
2 Holding both the holding faces (49) of the loader (20) with one hand from outside of the transparent cover (39).
3 Pushing in the graduated tube (40) till its proximal end (23) is slightly ahead of front opening (18) of tubular passage (9b) of pusher means (2).
4 Pushing in the push rod (50) in the graduated tube (40) such that the IUD (30) starts entering the negotiation passage (9a). The push rod (50) is pushed inside till the smooth curve (36) of the IUD (30) just starts projecting out from the first opening (12) from the stopping face (7) of the loader (20).
   At this instance, a root (52a) of the handle (52) of the solid rod (50) touches a distal end (28) of the graduated tube (40) and at this position the handle (52) of the solid rod (50) attains a position P2.
   Alternatively, either of the conditions, that is, either the projecting IUD (30) or the P2 position are achieved to signify and ensure that the arms (32n) of the IUD (30) are in folded condition and ready to be held in the graduated tube (40).
5 Advancing the graduated tube (40) towards the first opening (12) of the guide means (1) with a force ‘F’. The graduated tube (40) enters the negotiation passage (9a) and accommodates the arms (32) and the stem (33) of the IUD (30) at the proximal end (23) of the graduated tube (40).
6 Pushing the graduated tube (40) along with the IUD (30) with folded arms (32n) further towards the first opening (12) provided at the front end (11) of the guide means (1) such that an orifice (8) at a proximal end (23) of the graduated tube (40) comes out of the first opening (12) along with a captured IUD (30) out of the loader (20).

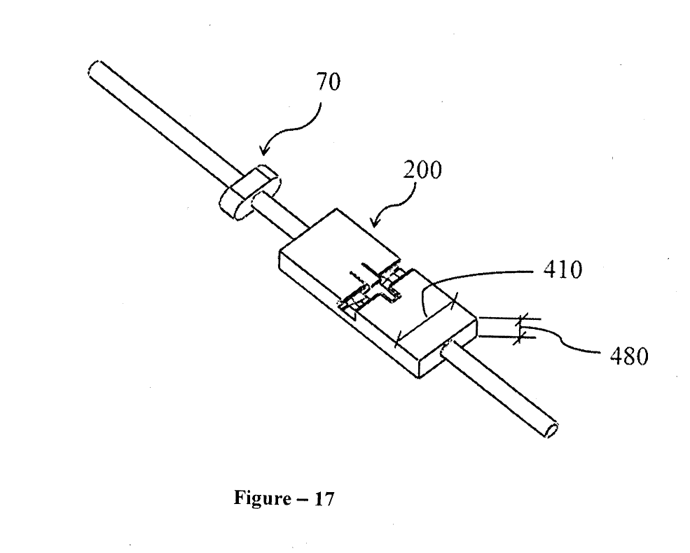

As another embodiment, the stopping face (7) is in the form of a separate component (70) as shown in FIG. 17; in which case there is no dimensional constraint on the width (410) and height (480) of the loader (200).

As another embodiment, the measurement marks (43) may not be there on the graduated tube (40) and the measurement may be done with a separate measurement device.

As another embodiment the guide means (1) of the loader (20) and the pusher means (2) of the loader (20) can be separate components as shown in FIG. 20.

We claim,

1. An apparatus for loading, insertion and placement of an intrauterine contraceptive device (IUD) in uterus, comprising:
a loader with a passage having a first opening and a second opening running along its elongated body;
a graduated tube mounted onto loader through its second opening;
a push rod running inside the graduated tube;
an IUD in an expanded condition parked in the loader;
in a sterile pouch having a transparent cover and a base holding the loader having the IUD parked and slidably mounted onto the graduated tube along with the push rod.

2. The apparatus as claimed in claim 1, wherein said loader is slidably mounted onto the graduated tube.

3. The apparatus as claimed in claim 1, wherein said loader has a length dimension of a guide means coordinated with the differential distance between a distal end of the graduated tube and a root of a handle of the push rod, when a tip of the push rod touches a bulging end of the IUD, in order to achieve a position P2 when the IUD just starts projecting out from the first opening of the loader.

4. The apparatus as claimed in claim 1, wherein said IUD is parked at a platform of the loader.

5. The apparatus as claimed in claim 1, wherein said graduated tube has a measurement markings provided on its outer surface.

6. The apparatus as claimed in claim 1, wherein said push rod has a localized projection to facilitate retention of the push rod inside the graduated tube.

7. The apparatus as claimed in claim 1, wherein said loader has a stopping face at its front end facilitates as a measurement reference and as a stopper.

8. The apparatus as claimed in claim 7, wherein said stopping face forms a part of the loader.

9. The apparatus as claimed in claim 7, wherein said stopping face is provided as a separate component.

10. A loader for loading of an intra-uterine contraceptive device (IUD) having an arms and a stem, in graduated tube, comprising:
a guide means has a first opening at a stopping face, forming a front portion of the loader;
a pusher means has a second opening at its rear end, forming a rear portion of the loader;
a platform connects the guide means of the loader to the pusher means of the loader;
a passage running from the first opening of the guide means to the second opening of the pusher means along an elongated body of the loader; and
a pair of C shaped exits on either side at the rear end of the guide means of the loader onto which the arms of an IUD rests while in an expanded condition.

11. A loader for loading of an intra-uterine contraceptive device (IUD) having an arms and a stem, in graduated tube, comprising:
a guide means has a first opening at a stopping face, forming a front portion of the loader;
a pusher means has a second opening at its rear end, forming a rear portion of the loader;
a platform connects the guide means of the loader to the pusher means of the loader;
a passage running from the first opening of the guide means to the second opening of the pusher means along an elongated body of the loader;
an IUD retainer in a vicinity at a rear end of the guide mean; and
a pair of C shaped exits on either side at the rear end of the guide means of the loader onto which the arms of an IUD rests while in an expanded condition.

12. The loader as claimed in claim 11, wherein said IUD retainer has a cantilever formed by providing slits on an upper surface of the loader towards pusher means.

13. The loader as claimed in claim 12, wherein said cantilever has teeth at its free end.

14. The loader as claimed in claim 13, wherein said teeth at its edge has an angle of 20 degree to 70 degree with respect to the platform.

15. The loader as claimed in claim 10 or claim 11, wherein said passage in the guide means begins from the pair of C shaped exits and converges to the first opening forming a negotiation passage.

16. The loader as claimed in claim 15, wherein said negotiation passage has a converging curved shape.

17. The loader as claimed in claim 15, wherein said negotiation passage has a straight and an angular slant shape.

18. The loader as claimed in claim 10 or claim 11, wherein said passage in the pusher means forms a tubular passage.

19. The loader as claimed in claim 10 or claim 11, wherein said first opening of the guide means has an area of cross section substantially same as an area of cross section of second opening of the pusher means.

20. The loader as claimed in claim 10 or claim 11, wherein said first opening has a non-circular cross section having a top that is flattened and a bottom slightly projected.

21. The loader as claimed in claim 10 or claim 11, wherein said first opening has a circular cross section.

22. The loader as claimed in claim 10 or claim 11, wherein said stopping face is a measurement reference for a sounding and a stopper.

23. The loader as claimed in claim 10 or claim 11, wherein said guide means of the loader and said pusher means of the loader are separate components.

24. The loader as claimed in claim 10 or claim 11 wherein the loader has an overall width and an overall height constrained to a minimum size, which can easily enter a vaginal cavity, after the vaginal cavity is stretched open for the IUD implantation, the overall width of the loader is less than 18 mm and the overall height of the loader is less than 12 mm.

25. A process of loading an IUD using a loader, the process comprising the steps of:
separating, partially, a transparent cover from a base of a sterile pouch, to be able to access and hold a handle of a push rod and a graduated tube.
holding a holding faces of a guide means, forming a front portion of the loader or a holding faces of a pusher means, forming a rear portion of the loader with one hand;
pushing the graduated tube, till its proximal end is slightly ahead of a front opening of a tubular passage of the pusher means of the loader;
pushing the push rod in the graduated tube;
advancing the graduated tube towards a first opening at a front end of the guide means with a force ‘F’ leading to an entry of the graduated tube into a negotiation passage;
accommodating an arms and a stem of the IUD at a proximal end of the graduated tube;
pushing the graduated tube outside the first opening at the front end of the guide means such that the graduated tube comes out of the first opening along with a captured IUD.

26. A process of loading an IUD using a loader, the process comprising the steps of:
separating, partially, a transparent cover from a base of a sterile pouch, to be able to access and hold a handle of a push rod and a graduated tube.
holding a holding face of the guide means or a holding faces of the pusher means with one hand;
pushing the graduated tube, till its proximal end touches an edge of a teeth of a cantilever of an IUD retainer;
pushing the push rod in the graduated tube;
advancing the graduated tube towards a first opening at a front end of a guide means of the loader, with a force ‘F’ leading to an entry of the graduated tube into a negotiation passage;
accommodating an arms and a stem of the IUD at a proximal end of the graduated tube;
pushing the graduated tube outside the first opening at the front end of the guide means such that the graduated tube comes out of the first opening along with a captured IUD.

27. The process of loading as claimed in claim 26, wherein, said teeth of the cantilever acts as an initial stopper for the graduated tube.

28. The loader as claimed in claim 26, wherein said teeth of the cantilever prevents the stem of the IUD from bending during a loading process.

29. The process of loading as claimed in claim 26, wherein advancing the graduated tube with the force ‘F’ acting on the edge of the teeth leads to a lifting up of the cantilever providing a way for the graduated tube into the negotiation passage.

30. The process of loading as claimed in claim 26, wherein advancing the graduated tube into the negotiation passage allows the arms and the stem of the IUD to enter the graduated tube as the arms and the stem of the IUD are substantially in a same orientation.

31. The process of loading as claimed in claim 25 or claim 26, wherein said pushing of the push rod, pushes a bulging end of the IUD resting on a platform to enter into the negotiation passage.

32. The process of loading as claimed in claim 31, wherein entering of the IUD into the negotiation passage allows the IUD to negotiate its shape and initiates folding of the arms of the IUD.

33. The process of loading as claimed in claim 25 or claim 26, wherein said pushing of the push rod allows the handle of the push rod to change from a position P to a position P1.

34. The process of loading as claimed in claim 25 or claim 26, wherein said pushing of the push rod is to visualize a smooth curve of the IUD when it just starts projecting out from the first opening.

35. The process of loading as claimed in claim 25 or claim 26, wherein said pushing of the push rod is to touch a root of the handle of the solid rod to a distal end of the graduated tube to attain a position P2.

36. The process of loading as claimed in claim 25 or claim 26, wherein said pushing of the push rod is to visualize of a smooth curve of the IUD when it just starts projecting out from the first opening and touch a root of the handle of the solid rod to a distal end of the graduated tube to attain a position P2.

37. The process of loading as claimed in claim 25 or claim 26, wherein the loading, that is, folding of arms of IUD is such that the folded arms are towards the upper surface of the loader and the stem of the IUD is towards the lower surface of the loader.

38. A process of insertion and placement of an IUD in a uterus, the method comprising the steps of:
adjusting a stopping face of the loader on measurement markings of the graduated tube on its outer surface;
removing a loaded apparatus i.e. the graduated tube along with the captured IUD in the orifice at its proximal end coming out of a first opening of the loader, from the pouch for a process of insertion;
inserting the loaded apparatus till the stopping face touches an external cervical os of the uterus;
withdrawing the graduated tube gradually, keeping the push rod steady, to first free the arms of the IUD from the graduated tube;
withdrawing the push rod along with the graduated tube completely from the uterus.
